(19) United States
(12) Patent Application Publication
(10) Pub. No.: US 2016/0074204 A1
(43) Pub. Date: Mar. 17, 2016

(54) LEG ENGAGING DEVICE AND METHOD

(71) Applicant: Todd Schragen, Livingston, NJ (US)

(72) Inventor: Todd Schragen, Livingston, NJ (US)

(21) Appl. No.: 14/483,279

(22) Filed: Sep. 11, 2014

(51) Int. Cl. A61F 5/37

(52) U.S. Cl. CPC A61F 5/37

(57) ABSTRACT

A device for engaging a leg near the knee includes a cradle having a lateral portion with a lateral axial length, a medial region, and a dorsal portion connecting between the lateral section and the medial region. The lateral portion extends distally farther than the dorsal portion. A fastening arrangement can secure the cradle to the leg. The lateral portion of the cradle is placed against the lateral side of the thigh and the dorsal portion is placed behind the thigh so that the lateral portion but not the dorsal portion laps the knee.

### BACKGROUND OF THE INVENTION

1. Field of the Invention

The present invention relates to techniques for engaging the leg, and in particular, to devices and methods for confining, restricting or guiding the leg.

2. Description of Related Art

The hip joint has several degrees of freedom. In particular, the leg (and femur) can rotate about its axis clockwise and counterclockwise relative to the hip socket. Rotation that points the kneecap away from the sagittal plane is deemed external rotation, while the reverse is internal rotation. Angular translation of the leg through a plane that swings the knee away from other leg is deemed abduction, the reverse is deemed adduction. Lifting of the knee engages the hip flexor.

Automobile drivers can suffer discomfort from remaining seated for extended time periods. The driving posture typically applies pressure to the gluteus maximus and tenses the hamstring. Since the right leg is active while driving (for countries that drive on the right), this seating posture is asymmetrical. Also, the angle of the femur relative to the spine can adversely affect the hip flexor. In particular, if this angle tends to be more 90 degrees or more the driver must expend more effort lifting the thigh and thereby stressing the hip flexor. In addition, repeatedly moving the foot between the gas pedal to the brake pedal typically causes hip abduction/abduction, and external/internal rotation at the hip joint. Specifically, movement from the brake pedal to the gas pedal usually involves external rotation and abduction at the hip joint (and the reverse for movement to the brake pedal). These motions, especially external rotation at the hip joint, can cause irritation of the driver's sciatic nerve (e.g., piriformis syndrome), as well as irritation of associated ligaments and other connective tissues.

See U.S. Pat. Nos. 1,753,367; 2,100,737; 2,522,887; 2,636,550; 2,650,654; 3,106,425; 3,173,722; 3,268,257; 3,679,262; 3,773,382; 4,105,249; 4,324,431; 4,334,709; 4,541,669; 4,583,781; 4,629,248; 4,673,216; 4,693,513; 4,696,516; 4,775,185; 5,018,790; 5,029,939; 5,152,578; 5,362,128; 5,707,108; 5,906,413; 7,255,396; 7,413,255; 7,578,554; 7,597,398; 7,603,913; 7,669,928; 7,819,474; and 8,282,171; as well as US Patent Application Publication Nos. 2001/0040397; 2003/0006639; 2006/0250007; 2008/0073959; 2009/0188045; 2011/0031792; 2011/0215200; 2011/0298261; 2012/0126604; and 2013/0020851.

### SUMMARY OF THE INVENTION

In accordance with the illustrative embodiments demonstrating features and advantages of the present invention, there is provided a device for engaging a leg near the knee. The device includes a cradle having a lateral portion with a lateral axial length, a medial region, and a dorsal portion connecting between the lateral section and the medial region, the lateral portion extending distally farther than the dorsal portion.

In accordance with another aspect of the invention, there is provided a device for engaging a leg near the knee. The device includes a cradle having a lateral portion with a lateral axial length, a medial region, and a dorsal portion connecting between the lateral section and the medial region. The device also has a fastening arrangement for securing the cradle to the leg.

In accordance with yet another aspect of the invention a method is provided for engaging a leg at the thigh and near the knee with a cradle having a medial region, a lateral portion, and between them a dorsal portion that extends distally less than the lateral portion. The method includes the step of placing the lateral portion of the cradle against the lateral side of the thigh and the dorsal portion behind the thigh with the lateral portion but not the dorsal portion lapping the knee.

By employing apparatus and methods of the foregoing type, an improved technique is achieved for restricting and guiding the leg to prevent discomfort or injury during activities such as driving. In a disclosed embodiment, a curved cradle is ergonomically shaped to fit around the thigh primarily on the lateral and dorsal sides of the thigh, but still extending somewhat into the medial region. The cradle can be secured to the thigh by a strap and can be used while driving.

In the disclosed embodiment, a lateral portion of the cradle has a distal, knee-lapping section that embraces the outside of the knee. The dorsal portion of the cradle is shorter and does not extend into the knee region and thus provides clearance for the calf. Also, an indented corner in the medial region provides additional clearance for avoiding discomfort in the groin.

If used while driving, the device tends to restrict the driver's ability to use external rotation to move from the brake pedal to the gas pedal. Such external rotation would tend to twist the thigh and swing the lower leg pendulum-like. Such external rotation will be impaired because the distal, knee-lapping section will tend to restrict such rotation. Basically, a driver attempting external rotation would feel a pinching sensation and would soon learn to move between pedals by means of abduction and adduction at the hip joint. Also, avoiding external rotation tends to keep the foot aligned in a vertical plane and not skewed across the gas pedal, which skewing can induce unnecessary stress in the foot and ankle.

### BRIEF DESCRIPTION OF THE DRAWINGS

The above brief description as well as other objects, features and advantages of the present invention will be more fully appreciated by reference to the following detailed description of illustrative embodiments in accordance with the present invention when taken in conjunction with the accompanying drawings, wherein:

FIG. 1 is a perspective view of a device in accordance with principles of the present invention;

FIG. 2 is an elevational, axial view of the distal end of the device of FIG. 1 with the fastening arrangement removed for simplification purposes;

FIG. 3 is a left side, elevational view of the device of FIG. 1 with the fastening arrangement removed;

FIG. 4 is a right side, elevational view of the device of FIG. 1 with the fastening arrangement removed;

FIG. 5 is a top view of the device of FIG. 1 with the fastening arrangement shown in phantom;

FIG. 6 is a bottom view of the device of FIG. 1 with the fastening arrangement shown in phantom;

FIG. 7A is a sectional view taken along line 7A-7A of FIG. 2;

FIG. 7B is a sectional view taken along line 7B-7B of FIG. 2;

FIG. 7C is a sectional view taken along line 7C-7C of FIG. 2;

FIG. 8 is perspective view showing the device of FIG. 1 secured to the leg of a driver of an automobile;

FIG. 9 is an elevational, axial view of the distal end of a device that is an alternate to that of FIG. 2;

FIG. 10 is a left side, elevational view of the device of FIG. 9;

FIG. 11 is a right side, elevational view of the device of FIG. 9;

FIG. 12 is a bottom view of the device of FIG. 9; and

FIG. 13 is a sectional view taken along line 113-13 of FIG. 9.

### DETAILED DESCRIPTION

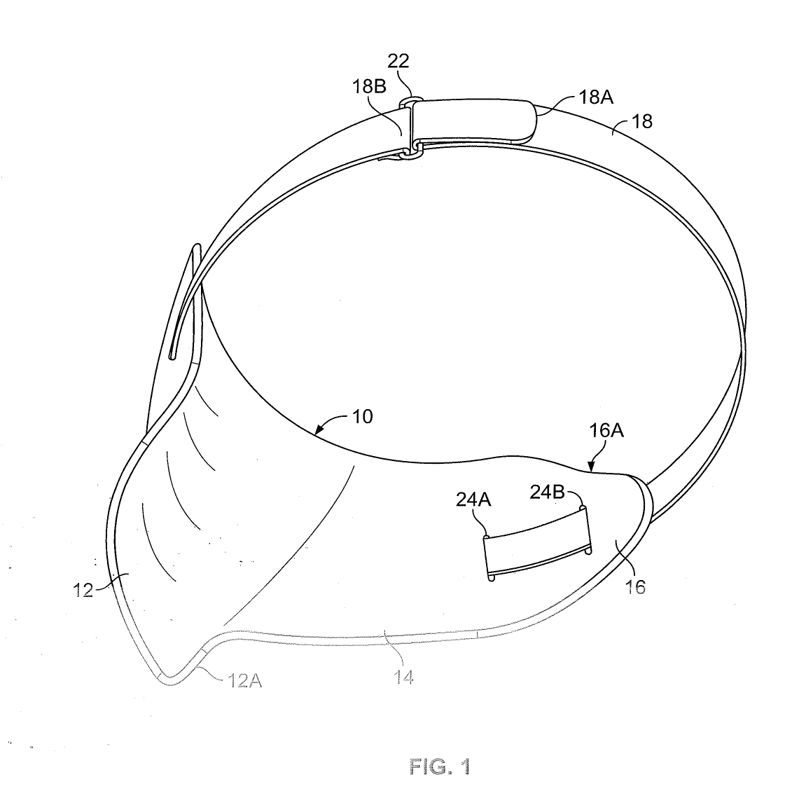

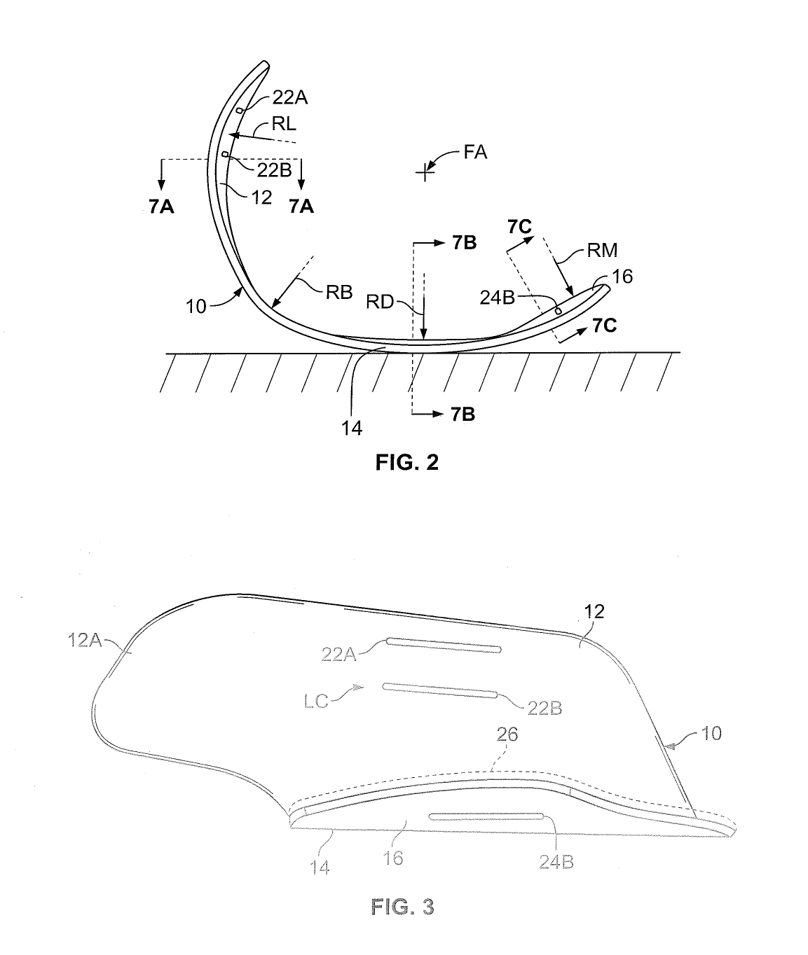

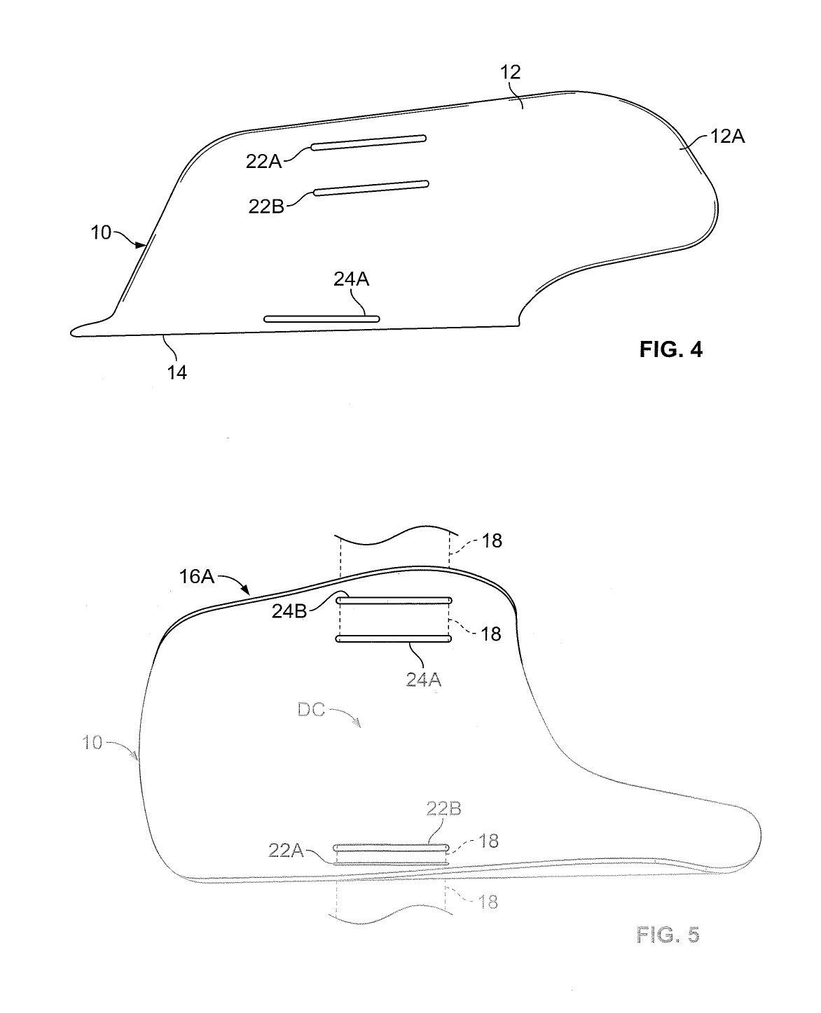

Referring to FIGS. 1-7, the illustrated device has a cradle 10 with a lateral portion 12, dorsal portion 14, and medial region 16. Cradle 10 may be an ergonomically curved panel designed to fit around the thigh of a human leg and extend axially, that is, extend along the axis of the femur (femoral axis FA of FIG. 2). Cradle 10 may be an ⅛ inch (3 mm) thick thermoplastic panel, although other embodiments will use a different thickness and a material other than a thermoplastic. FIG. 2 should be considered an end view of cradle 10 as well as an axial view, that is, a view along the axis FA of the femur.

As described further hereinafter, cradle 10 is designed to embrace the right thigh of a person seated in a driver's seat, in which case the back (dorsum) of the thigh is flattened somewhat relative to the right (lateral) side and the left (medial) side of the thigh. It will be appreciated that in some cases, especially for countries that drive on the left, the device will be applied to the left leg and may therefore be rendered as the mirror image of that shown in the drawings.

The transition from the lateral section 12 to dorsal section 14 may be distinguished by their different radiuses of curvature. For example, the radius of curvature RL of lateral section 12 will, for the most part, be much smaller than the radius of curvature RD of dorsal section 14 over most of its arc. These radiuses of curvature are defined along a transection line defined by the intersection of the inside surface of the cradle 10 with a plane perpendicular to femoral axis FA. Radius of curvature is measured in the usual way, that is, the radius of the arc best matching the curvature at the point of measurement. Also, the foregoing radiuses of curvature are sometimes referred to as circumferential radiuses of curvature.

In one embodiment designed for the average adult male anatomy, the radius of curvature RL at a central inside surface LC (FIG. 3) of lateral section 12 was approximately 1½ inches (3.8 cm), while the radius of curvature RD at a central inside surface DC (FIG. 5) of dorsal section 14 was two to three times greater; for example, 4 inches (10 cm). It will be appreciated that these radiuses can be varied to account for different weights, heights, ages, genders, and atypical variations

These radiuses of curvature are designed to achieve a comfortable fit on the lateral and dorsal sides of a thigh of a seated person. With this in mind, sections can be approximately and conveniently defined by adjusting the device 10 for a most comfortable feel and then defining the center of the lateral section 12 as that aligned with the leg's outermost lateral position (viewed perhaps as the 3 o'clock position), while the center of the dorsal section 14 can be viewed as that aligned with the leg's rearmost dorsal position (viewed perhaps as the six o'clock position). In this case the transition between lateral section 12 and dorsal section 14 can be viewed as the halfway point (viewed perhaps as the 4:30 clock position).

One can arrive at approximately the same conclusion by defining the transition between the lateral section 12 and dorsal section 14 by an inverse ratio, that is, the transition occurs where the radius of curvature RB satisfies the following relationship: RB/RL=RD/RB. It will be understood that the radius of curvature will, in most embodiments, vary continuously throughout the arc of cradle 10.

In case one wishes to design cradle 10 more abstractly, a hypothetical cross-sectional outline of a typical adult male thigh, halfway down the femur, may be approximated as an ellipse with a major axis that is 6¾ wide (17 cm) long and a minor axis that is 5.0 inches (12.7 cm) deep. In this case, the femoral axis will be considered the intersection of the major and minor axis.

Of course these elliptical dimensions can be varied to account for different weights, heights, ages, genders, and atypical variations. While this hypothetical thigh can be approximated as a right elliptical prism, better results are achieved with a hypothetical solid that is tapered in a manner to simulate human anatomy.

Regarding the medial region 16, it extends medially from dorsal section 14 and does not necessarily embrace most, much, or even any of the medial region of the thigh. In the embodiment of FIG. 2 the medial region 16 and dorsal section 14 extend further in the medial direction than the lateral direction, which is unsurprising since coverage in the lateral direction is accomplished by lateral section 12.

Good results can be achieved when the dorsal section 14 and medial region 16 extend less than 90°, as measured with respect to femoral axis FA. Also, good results can be achieved when the lateral section 12 and dorsal section 14 encompass more than 90°, again as measured with respect to femoral axis FA. Furthermore, good results can be achieved when cradle 10 (intervals 12, 14 and 16) extends more than 120°, again as measured about femoral axis FA.

It will be noticed that lateral section 12 extends distally farther than dorsal section 14 and forms cantilevered distal section 12A. Distal section 12A is designed to lap the knee, basically reaching the lateral side of the knee (right side in this embodiment). Section 12A is flared outwardly to prevent unnecessary pressure and discomfort at the knee and adjacent portions of the thigh. In contrast, dorsal section 14 is designed to avoid intersecting the knee and provides clearance for the calf. The width of distal section 12A varies somewhat along its length but this width (between its anterior and posterior edge) is approximately 3½ inches (9 cm), but can be varied depending upon the user's anatomy or preferences.

Lateral section 12 has a lateral axial length greater than the dorsal axial length of dorsal section 14. The lateral (dorsal) axial length is the length measured along a line defined by a plane containing femoral axis FA that intersects a central inside surface LC (DC) of section 12 (14).

The greatest axial length occurs in lateral section 12 to establish an overall axial length of 11½ inches (29 cm). The axial length of dorsal section 14 at central region DC is 7¼ inches (18.4 cm). An overall width of 6¾ inches (17 cm) is measured from the anterior edge of lateral section 12 to the outermost edge of medial region 16. The elevation of the anterior edge of lateral section 12 relative to central surface DC is 3¾ inches (9.5 cm). If cradle 10 were laid flat on a horizontal surface with dorsal section 14 up, the overall height would be 3 inches (7.6 cm). It will be understood that the foregoing dimensions are exemplary and can be changed in other embodiments to satisfy a user's preference. Moreover, these dimensions can be varied to accommodate different sizes of thighs associated with users of different height, weight, age, gender, etc.

Medial region 16 has an indented inside corner 16A. As explained further hereinafter, this indentation provides clearance in the groin region.

In some embodiments the inside of cradle 10 can be lined with a pad. In particular, an inside pad 26 is shown in phantom in FIG. 3. In some embodiments the pad 26 may be a simple polyurethane foam. In other embodiments pad 26 may be a gel pad overlying a styrofoam backer. In still other embodiments pad 26 may be a commercially available memory foam.

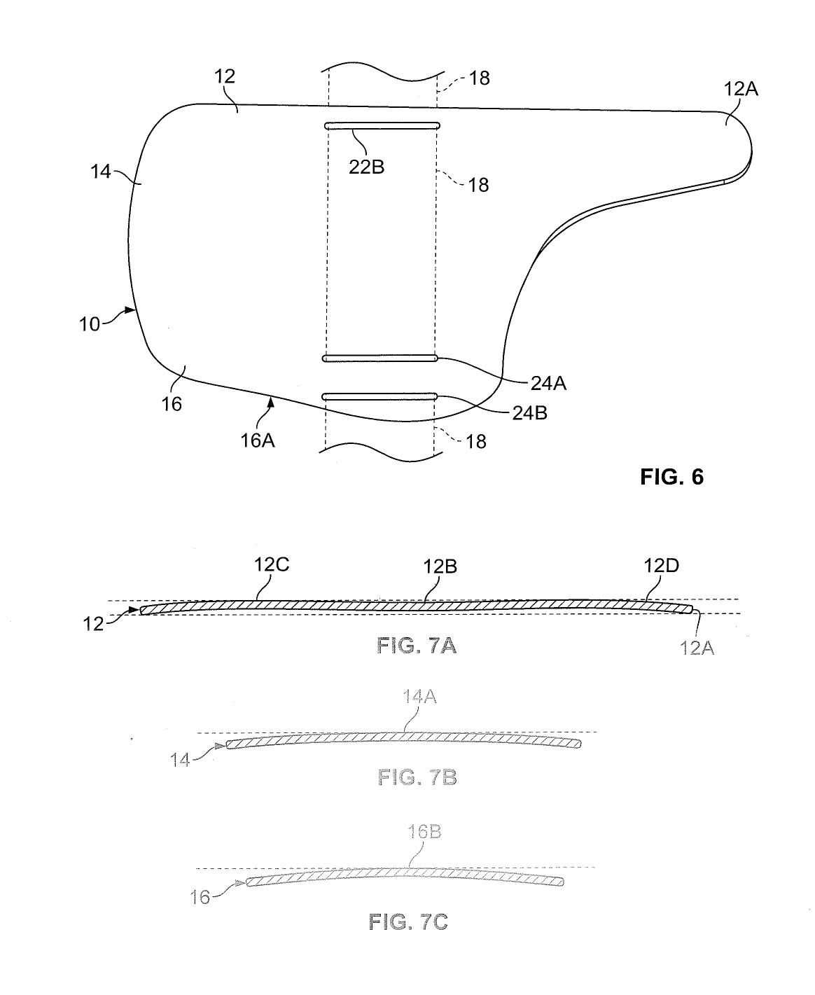

As shown most clearly in FIG. 6, the inside surface of cradle 10 along intervals 12, 14, and 16 remains concave in the circumferential direction, that is, in a direction defined by angular displacement relative to femoral axis FA. From FIG. 7A the curvature of the lateral section 12 varies along the axial direction, that is, along a direction parallel to femoral axis FA. Specifically, inside surface 12B is concave but is bordered by convex surfaces 12C and 12D. To reveal this curvature more clearly, the section in FIG. 7A of lateral portion 12 is drawn between two arbitrary, parallel, dashed lines.

As shown in FIGS. 7B and 7C the inside surfaces 14A and 16B of the sections 14 and 16, respectively, are convex. Dashed, tangential line lines are shown in FIGS. 7B and 7C to reveal this curvature more clearly. As explained further hereinafter, the convexity of dorsal section 14 provides the benefit of elevating the thigh slightly.

A fastening arrangement is shown herein as a releasable strap 18 that is threaded through slots 22A, 22B, 24A, and 24B in cradle 10. Specifically free end 18A is threaded under lateral section 12 and into slot 22A. From there, free end 18A is threaded through slot 22B to the outside of lateral section 12. Thereafter, free end 18A is threaded behind dorsal section 14 and through slot 24A to the inside of cradle 10. Finally, free end 18A is threaded through slot 24B before being threaded through buckle 22 and attached to end 18B of strap 18.

In this embodiment, except for the last 3 inches (7.6 cm) of free end 18A, the outside of strap 18 has loop material that is part of a hook and loop fastener. This loop material can fasten to the hook material on the opposing face of free end 18A.

Specifically, end 18A is threaded through buckle 22 and lapped back on to the outside of strap 18. In FIG. 1, free end 18A is shown secured to strap 18 a short distance from buckle 22. In more adjustable and secure embodiments free end 18A is secured at a much greater distance. In one embodiment, free end 18A was wound completely around the outside of cradle 10, before being secured to the outside of strap 18, short of buckle 22.

Instead of hook and loop fasteners, a variety of alternate buckling arrangements can be used. Also, in some cases the strap can be secured by snaps or can be tied into a loop. In some embodiments strap 18 may be elastic to provide a more accommodating fit.

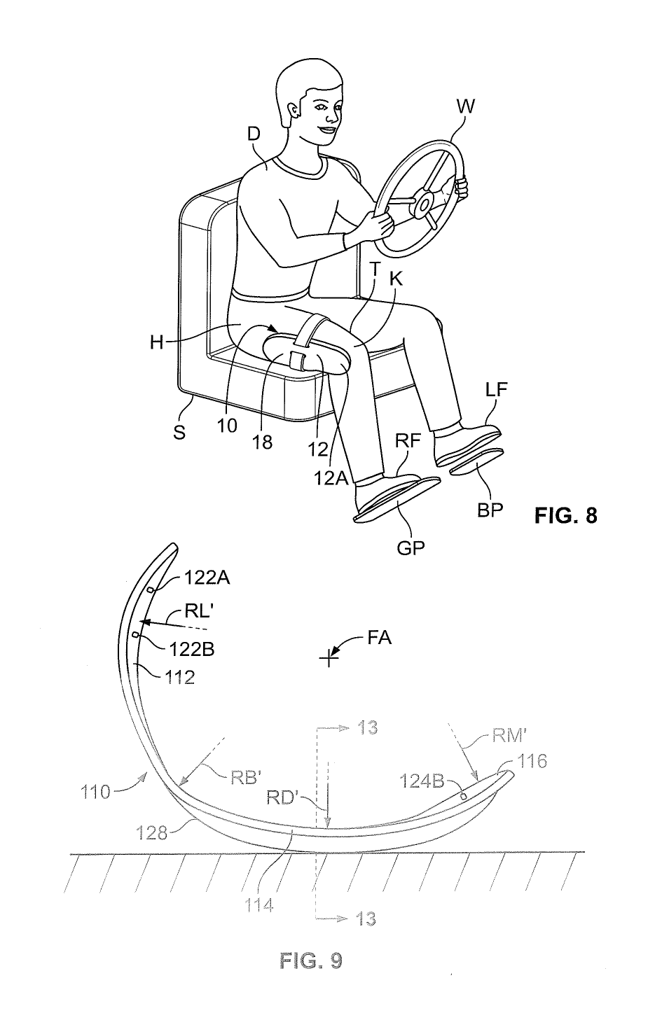

To facilitate an understanding of the principles associated with the foregoing apparatus of FIGS. 1-7, its operation will be briefly described with reference to FIG. 8. With belt 18 unfastened and free end 18A removed from buckle 22, driver D may install cradle 10 on thigh T, before or after sitting in car seat S. In some cases belt 18 may instead be simply loosened so that driver D can step into the device and pull it up to the thigh T.

In any event, dorsal section 14 will be placed underneath thigh T with distal section 12A lapping knee K on the lateral side as shown in FIG. 8. The tip of section 12A may extend as far as, or almost as far as, the knee cap. Also, cradle 10 may be rotated to lift or lower section 12A as desired. The position of section 12A will be adjusted to embrace and restrict the knee K comfortably, according to the driver's preferences. It will be noticed that the convex portion 12D (FIG. 7A) of lateral section 12 outwardly flares section 12A to prevent excessive pressure on knee K. Also, indented corner 16A prevents pressure and discomfort at the groin region.

Once positioned appropriately in this manner, the driver D may secure cradle 10 to thigh T by inserting free end 18A through buckle 22 and tightening strap 18. Strap 18 kept tight by securing the hook material on free end 18A to the loop material on the outside of strap 18. Driver D is now prepared to drive, and is shown in FIG. 8 gripping steering wheel W, and depressing gas pedal GP with right foot RF.

Once in place, the device 10/18 tends to restrict the driver's ability to use external rotation to move from the brake pedal BP to the gas pedal GP. Such external rotation would tend to axially twist thigh T and swing the lower leg and foot RF pendulum-like. Such external rotation at the hip joint H, can cause irritation of the driver's sciatic nerve (e.g., piriformis syndrome), as well as irritation of associated ligaments and other connective tissues.

However, attempts at external rotation will be impaired because the distal, knee-lapping section 12A will tend to restrict such rotation. Basically, if external rotation is attempted driver D would feel a pinching sensation from section 10A and would soon learn to move between petals BP and GP by means of abduction and adduction at the hip joint H.

Also, avoiding external rotation tends to keep the foot RF in a vertical plane and not skewed across the gas pedal GP, which skewing can induce unnecessary stress in the foot and ankle.

In addition, the convexity 14A (FIG. 7B) of dorsal section 14 tends to lift thigh T to relieve stress on the hip flexor.

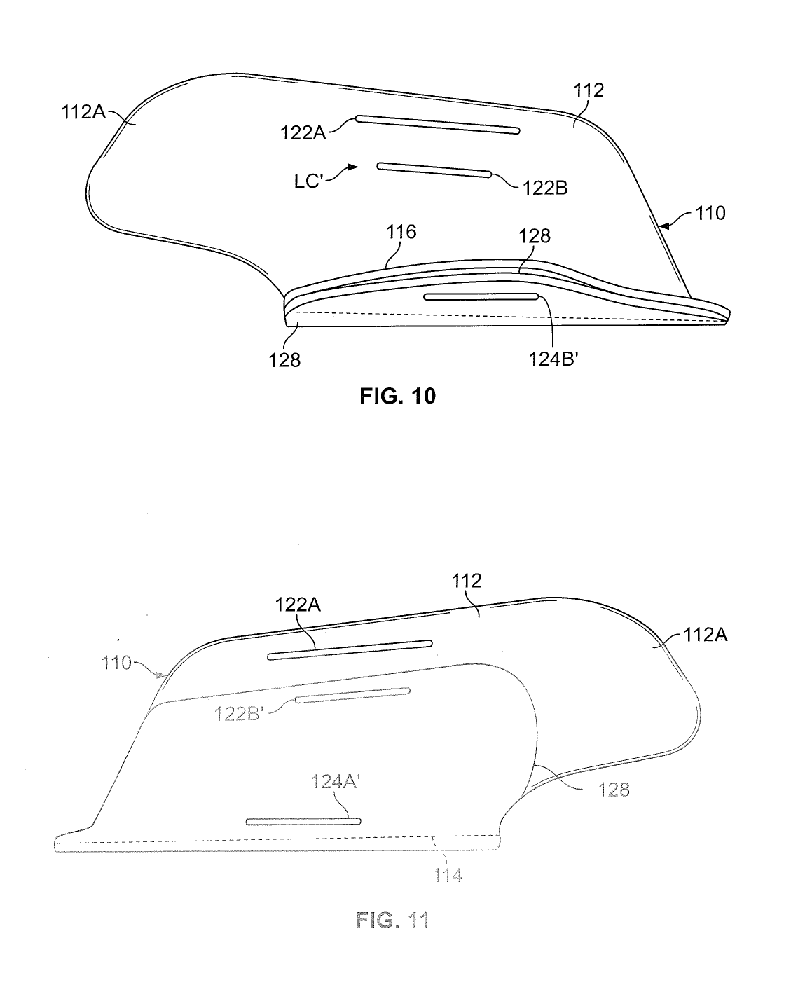

Referring to FIGS. 9-13, the illustrated device is similar to the cradle previously described in FIGS. 1-8. Components corresponding to those of FIGS. 1-8, have the same reference numerals but increased by 100. Previously mentioned radiuses of curvature are similar as well, and have been marked by a prime (′) to appear as radiuses RL′, RB′, RD′ and RM′.

As before, cradle 110 has a lateral portion 112, dorsal portion 114, and medial region 116 designed to fit around the thigh of a human leg and extend axially along femoral axis FA. Cradle 110 uses similar materials and may have the same overall dimensions. FIG. 9 is an axial view along the axis FA of the femur.

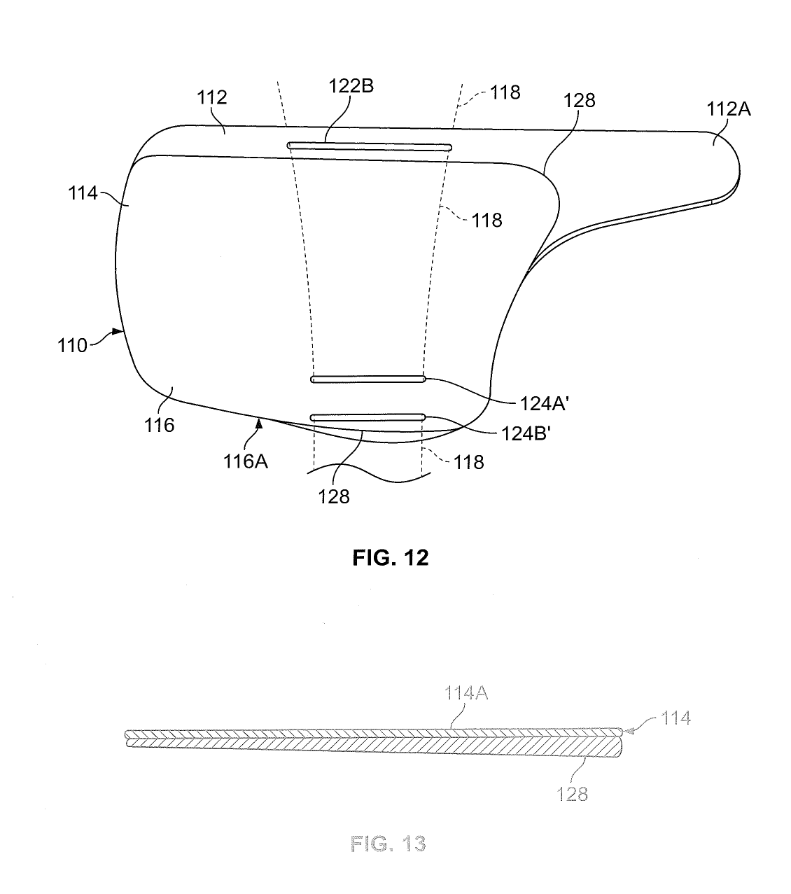

As shown in FIG. 13 the inside surface 114A of the dorsal section 114 remains straight in the axial direction. However, the thickness of the dorsal section 114 varies and increases in the distal direction. This increasing thickness is achieved by employing a spacer 128 on the underside of the cradle 110. As shown in FIGS. 9 and 12, spacer 128 extends over all of the main dorsal section 114 and intrudes somewhat into medial section 116 and lateral section 112. In this embodiment spacer 128 is a pliable plastic layer attached to cradle 110 by adhesives, heat welding, or other means. Also, the distal end of spacer 128 is ⅝″ (16 mm) thick and tapers in the proximal direction to 1/16″ (1.6 mm), although these thicknesses and the tapering profile can be varied depending upon the user's needs and preferences.

In some cases cradle 110 is molded so that dorsal section 114 is an integral piece with a distally increasing thickness, thereby eliminating the need for a discrete spacer 128.

As before, cradle 110 has slots for receiving strap 118. Specifically, lateral section 112 has a pair of strap slots 122A and 122B, which do not intersect spacer 128. Spacer 128 does, however, intersect the pair of strap slots in the medial section 116 (only slot 124B being visible in FIG. 9). The strap slots in medial section 116 register with corresponding strap slots 124A′ and 124B′ in spacer 128. As before, strap 118 is laced to the inside between slots 122A and 122B, and between slots 124A′ and 124B′.

In this embodiment, the portion of strap 118 emerging from slot 122A (FIG. 12) is wider than the portion emerging in the opposite direction from slot 124B. Slots 122A and 122B are lengthened to accommodate this increased strap width. This wider strap portion provides a larger area for comfortably engaging the anterior of the user's thigh. This wider portion of strap 118 emerging from slot 122A eventually tapers to terminate in a narrower strip (not shown) designed to engage and close on the opposite end of the strap. The opposite ends of strap 118 can be fastened together as previously describe.

The foregoing cradle 110 is worn by the user as previously described. The increased thickness at the distal end of dorsal section 114 elevates the thigh and knee, thereby taking pressure off the hip flexor. This added thickness also increases the downward pressure at the distal end of dorsal section 114 thereby reducing the tendency toward external rotation of the thigh.

It is appreciated that various modifications may be implemented with respect to the above described embodiments. The foregoing cradle may be made of relatively stiff materials or materials that can flex somewhat but still tend to urge the leg into the desired position. In some cases the cradle may be heat deformable so that the user can manually adjust the cradle's configuration after purchase. In other cases the cradle may be formed of a flexible material but with relatively rigid stays that impose a specific orientation on the user's leg. While a single encompassing strap is illustrated, in some embodiments the device may incorporate two or more straps. In some embodiments the cradle may be a clamshell device with halves that can be swung together on a hinge in order to encompass the thigh more fully. In other cases the device may be attached to a car seat by a tether or other mechanism in order to allow some abduction/abduction of the femur while still restricting external rotation at the hip joint. In still other embodiments the cradle may be incorporated into an elastic sleeve or the leg of a pair of driving pants that hold the cradle in position against the driver's leg. In some embodiments the cradle may be made of multiple components that can extend and retract relative to each other and thereby allow the user to adjust the cradle's configuration as desired.

Obviously, many modifications and variations of the present invention are possible in light of the above teachings. It is therefore to be understood that within the scope of the appended claims, the invention may be practiced otherwise than as specifically described.

1. A device for engaging a leg near the knee, the device comprising:
a cradle having a lateral portion with a lateral axial length, a medial region, and a dorsal portion connecting between said lateral section and said medial region, said lateral portion extending distally farther than said dorsal portion.

2. A device according to claim 1 wherein said dorsal portion has a dorsal axial length that is less than the lateral axial length.

3. A device according to claim 1 comprising:
a fastening arrangement for securing said cradle to said leg.

4. A device according to claim 3 wherein said fastening arrangement comprises a releasable strap.

5. A device according to claim 3 wherein said fastening arrangement comprises an elastic strap.

6. A device according to claim 1 wherein said cradle is shaped and sized to allow said lateral portion to reach the knee without said dorsal portion reaching the knee.

7. A device according to claim 1 wherein said lateral portion has an outwardly flared distal section.

8. A device according to claim 1 wherein said lateral portion has a circumferential radius of curvature less than that of most of said dorsal portion.

9. A device according to claim 1 wherein said lateral portion and said dorsal portion extend circumferentially more than 90°, said medial region and said dorsal region extending circumferentially less than 90°.

10. A device according to claim 9 wherein said lateral section, said dorsal section and said medial region extend circumferentially more than 120°.

11. A device according to claim 1 wherein said dorsal portion has a central inside surface that is circumferentially concave and axially convex.

12. A device according to claim 1 wherein said medial region has an indented proximal corner for providing groin clearance.

13. A device according to claim 1 comprising:
a pad internally lining said cradle.

14. A device according to claim 1 wherein said dorsal portion has a varying thickness that increases distally.

15. A device according to claim 1 comprising:
a spacer underlying said dorsal portion and having a thickness that increases distally.

16. A device for engaging a leg near the knee, the device comprising:
a cradle having a lateral portion with a lateral axial length, a medial region, and a dorsal portion connecting between said lateral section and said medial region; and
a fastening arrangement for securing said cradle to said leg.

17. A device according to claim 16′ wherein said fastening arrangement comprises a releasable strap.

18. A device according to claim 16 wherein said fastening arrangement comprises an elastic strap.

19. A device according to claim 16 wherein said cradle is shaped and sized to allow said lateral potion to reach the knee without said dorsal portion reaching the knee.

20. A device according to claim 16 wherein said lateral portion has an outwardly flared distal section.

21. A device according to claim 16 wherein said lateral portion has a circumferential radius of curvature exceeding that of most of said dorsal portion.

22. A device according to claim 16 wherein said lateral portion and said dorsal portion extend circumferentially more than 90°, said medial region and said dorsal portion extending circumferentially less than 90°.

23. A device according to claim 22 wherein said lateral section, said dorsal section and said medial region extend circumferentially more than 120°.

24. A device according to claim 16 wherein said medial region has an indented proximal corner for providing groin clearance.

25. A device according to claim 16 comprising:
a pad internally lining said cradle.

26. A device according to claim 16 wherein said dorsal portion has a varying thickness that increases distally.

27. A device according to claim 16 comprising:
a spacer underlying said dorsal portion and having a thickness that increases distally.

28. A method for engaging a leg at the thigh and near the knee with a cradle having a medial region, a lateral portion, and between them a dorsal portion that extends distally less than said lateral portion, the method comprising the step of:
placing the lateral portion of the cradle against the lateral side of the thigh and the dorsal portion behind the thigh with the lateral portion but not the dorsal portion lapping the knee.

29. A method according to claim 28 comprising the steps of:
fastening the cradle to the thigh.

30. A method according to claim 29 comprising the steps of:
sitting in a car seat with the cradle fastened to the thigh and the dorsal portion pressed against the car seat in order to restrict external rotation of the thigh at the hip when operating automotive pedals.

31. A method according to claim 30 wherein the cradle is fastened to the thigh in order to restrict abduction of the thigh at the hip.
